(19) United States
(12) Patent Application Publication
(10) Pub. No.: US 2026/0077116 A1
(43) Pub. Date: Mar. 19, 2026

(54) CARTRIDGE WITH LOCKING MECHANISM FOR LOCKING CARTRIDGE INSERT WITH INTEGRATED INFUSION NEEDLE AND CGM SENSOR

(71) Applicant: AITA BIO INC., New York, NY (US)

(72) Inventors: Dilan Casanovas Mack, New York, NY (US); Russell Cole, River Vale, NJ (US)

(21) Appl. No.: 19/106,766

(22) PCT Filed: Sep. 16, 2023
(86) PCT No.: PCT/US2023/032965

§ 371 Date: Feb. 26, 2025

Related U.S. Application Data
(60) Provisional application No. 63/415,402, filed on Oct. 12, 2022.

(51) Int. Cl. A61M 5/14; A61M 5/158; A61M 5/172

(52) U.S. Cl. CPC A61M 5/1413; A61M 2005/1585; A61M 2005/1726; A61M 2230/201

(57) ABSTRACT

An infusion system is disclosed comprising: a device for delivering insulin to a user, the device including a reservoir and a micropump for pumping the insulin from the reservoir into tissue of a user; and a cartridge for securing a cartridge insert that includes (a) an infusion needle configured to infuse the insulin and introduce a CGM sensor into the user or (b) an introducer needle for introducing an infusion catheter and the CGM sensor into the user, the cartridge configured to move from (1) a first position, wherein the infusion needle or an introducer needle is above the tissue of the user to (2) an second position, wherein the infusion needle or the introducer needle is in a deployed position inserted into tissue of the user, wherein the cartridge includes a locking mechanism to lock the cartridge insert into the cartridge.

### CROSS REFERENCE TO RELATED APPLICATIONS

This application claims priority to U.S. provisional application No. 63/415,402, filed Oct. 12, 2022, entitled “Needle Cartridge Assembly With Locking Mechanism For Locking Cartridge Insert With CGM Sensor” which is incorporated by reference herein.

### FIELD OF THE INVENTION

The present invention relates to a cartridge with locking mechanism for locking a cartridge insert with a integrated infusion needle and a CGM sensor.

### BACKGROUND OF THE INVENTION

Insulin pumps help people with diabetes to conveniently manage their blood sugar. These devices deliver insulin at specific times. Insulin patch pumps or pods are one type of insulin pump. The pods are wearable devices that adhere to the skin of a user using an adhesive patch. The pods deliver insulin from a chamber and internal cannula based on separately acquired CGM sensor readings.

It would be advantageous to provide improvements to insulin pumps described above.

### SUMMARY OF THE INVENTION

A cartridge is disclosed with a locking mechanism for locking a cartridge with an integrated introducer needle and CGM sensor.

In accordance with an embodiment of the present disclosure, an infusion system comprising: a device for delivering fluid to a user, the device including a reservoir and a micropump for pumping the fluid from the reservoir into a user; and a cartridge for supporting a cartridge insert that includes (a) an infusion needle configured to infuse the fluid and introduce an analyte sensor into the user or (b) an introducer needle for introducing an infusion catheter and the analyte sensor, the cartridge configured to move from (1) a first position, wherein the infusion needle or an introducer needle is in a non-deployed position outside of the user to (2) a second position, wherein the infusion needle or an introducer needle is in a deployed position inserted into tissue of the user, wherein the cartridge includes a locking mechanism to lock the cartridge insert into the cartridge assembly.

In accordance with another embodiment of this disclosure, an infusion system comprising: a device for delivering insulin to a user, the device including a reservoir and a micropump for pumping the insulin from the reservoir into tissue of a user; and a cartridge for securing a cartridge insert that includes (a) an infusion needle configured to infuse the insulin and introduce a CGM sensor into the user or (b) an introducer needle for introducing an infusion catheter and the CGM sensor into the user, the cartridge configured to move from (1) a first position, wherein the infusion needle or an introducer needle is above the tissue of the user to (2) an second position, wherein the infusion needle or the introducer needle is in a deployed position inserted into tissue of the user, wherein the cartridge includes a locking mechanism to lock the cartridge insert into the cartridge.

In accordance with yet another embodiment of the disclosure, An infusion system comprising: a device for delivering insulin to a user, the device including a reservoir and a micropump for pumping the insulin from the reservoir into tissue of a user; and a cartridge assembly including: (1) a cartridge insert that includes (a) an integrated infusion needle and CGM sensor or (b) an introducer needle for introducing an infusion catheter and a CGM sensor and (2) a cartridge for securing therein the cartridge insert, the cartridge assembly configured to move from (a) a telescoping position, wherein the integrated infusion needle and CGM sensor or an introducer needle, infusion catheter and CGM sensor are in a retracted position above the tissue of the user to (b) an advanced position, wherein the integrated infusion needle and CGM sensor or an introducer needle, infusion catheter and CGM sensor is in a deployed position inserted into tissue of the user, wherein the cartridge includes a locking mechanism to lock the cartridge insert into the cartridge.

### BRIEF DESCRIPTION OF DRAWINGS

FIGS. 1-2 depict perspective views of an infusion system for infusing insulin into a user in a pre-activation and post-activation configuration, respectively.

FIG. 3 depicts a perspective view of the infusion system in FIG. 2 wherein a device for delivering insulin is shown without a needle cartridge assembly.

FIGS. 4A-D depict various views of a needle cartridge assembly of the infusion system of FIGS. 1-2.

FIGS. 5 and 6 depict the needle cartridge assembly of the infusion system in FIGS. 1 and 2 in a telescoping position.

FIG. 7 depicts a front perspective view of the infusion system in FIG. 2 in which the needle cartridge assembly, in an advanced position, is exposed.

FIG. 8 depicts a cross sectional view (in part) of the infusion system of FIG. 2 in a post activation configuration in which the needle cartridge assembly has fully advanced in the device for delivering insulin of the infusion system.

FIG. 9 depicts a cross sectional view of a baseplate of the infusion system of FIG. 2.

FIG. 10 depicts the components of the infusion system of shown in FIG. 1 in an exploded configuration.

FIGS. 11-13 depict various cross-sectional views of the infusion system of FIG. 1 in a pre-activation configuration.

FIGS. 14-16 depict various cross-sectional views of the infusion system of FIG. 1 in a post activation configuration.

FIGS. 17-24 depict various views of an example cartridge assembly of another infusion system.

FIG. 25 depicts a block diagram of example components of the device for delivering insulin and needle cartridge assembly of the infusion system.

### DETAILED DESCRIPTION OF THE INVENTION

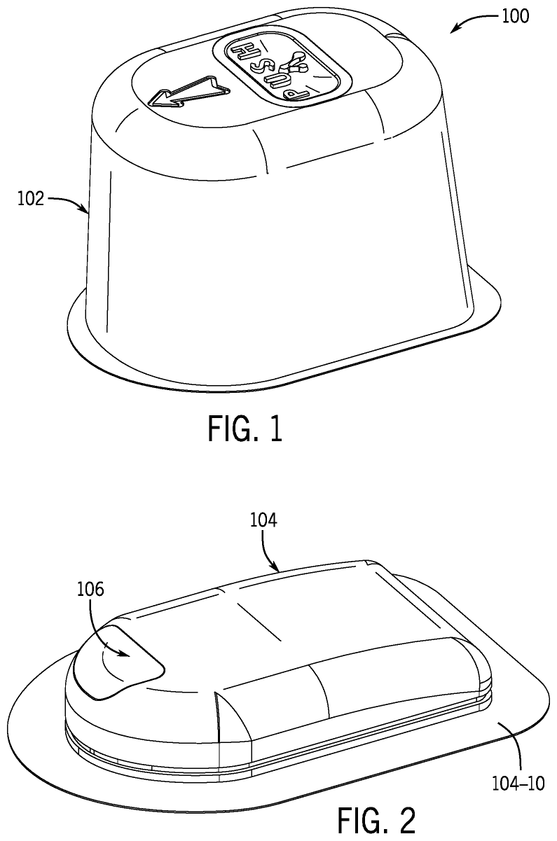

FIGS. 1 and 2 depict perspective views of infusion system 100 for infusing insulin (or other fluid medication) into a user (patient) in pre-activation and post activation configurations, respectively. Infusion system 100 comprises a detachable activation mechanism 102 and device 104 (or pod) for delivering insulin to the user. As described in more detail below, device 104 is a wearable apparatus, system or pod for diabetes management in which continuous glucose monitoring (CGM), insulin delivery and control functionality are provided to ensure insulin is delivered at very precise rates and has the capability of detecting occlusions in real time. In operation, device 104 is applied by opening a sterile packaging, filling the reservoir with insulin, priming the fluid path, removing the adhesive backing, sticking infusion system 100 to the desired body location, pushing a button assembly, removing and disposing the detachable activation mechanism 102. Removing detachable activation mechanism 102 components that are necessary for device 104 activation and needle insertion, but not required for infusion or sensing offers the benefit of a smaller, lower profile, more comfortable and discrete wearable device 104 for the user. This is described in more detail below.

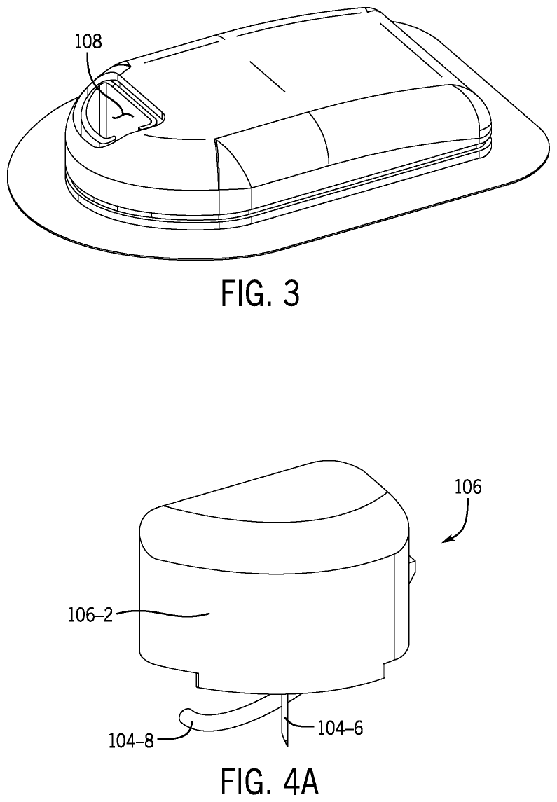

Detachable activation mechanism 102 is configured to insert an indwelling integrated infusion needle and continuous glucose monitoring (CGM) sensor (104-6 described below) of device 104 in a single insertion site normal to a user's skin or at other desired angles known to those skilled in the art. FIGS. 2 and 3 clearly show device 104, but FIG. 3 illustrates device 104 without needle cartridge assembly 106 (also referred to as cartridge 106). This is described in more detail below. Integrated infusion needle is preferably constructed of steel but it may be any rigid material known to those skilled in the art. In this embodiment described herein, detachable activation mechanism 102 and needle cartridge assembly 106 are described with respect to inserting an integrated infusion needle and CGM sensor. However, detachable activation mechanism 102 and/or needle cartridge assembly 106 may be used with an infusion needle or infusion catheter only (without CGM sensor capability or any other analyte sensor) as known to those skilled in the art.

Device 104 incorporates, among other elements (as described below), a micropump as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medication to the user (patient). Medication may include small molecule pharmaceutical solutions, large molecule or protein drug solutions, saline solutions, blood or other fluids known to those skilled in the art. Insulin is an example of fluid that is described in this application. However, micropump may be used in other environments known to those skilled in the art.

Device 104 also includes reservoir, a microcontroller unit (MCU) (not shown), integrated infusion needle and CGM sensor 104-6 and a battery and power controller (not shown). The reservoir is configured to receive and store insulin for its delivery over a course of about three days, or as needed. However, reservoir size may be configured for storing any quantity of fluid as required. The micropump fluidly communicates with reservoir to enable infusion as needed. CGM, as known to those skilled in the art, tracks patient glucose levels and permits those levels to be used in algorithms that control flow rate. CGM sensor is only one type of analyte sensor that may be used in the infusion system described herein. Other analyte sensors may be used or integrated with an infusion needle as known to those skilled in the art. MCU controls the operation of the micropump to deliver insulin through the insulin needle 104-6 from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU.

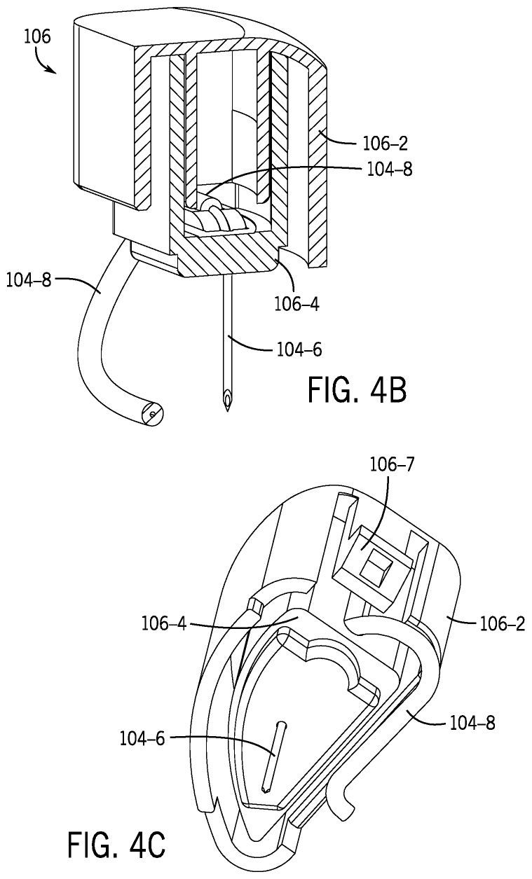

Infusion system 100 further incorporates needle cartridge assembly 106 as shown in FIGS. 4A-4D. Needle cartridge assembly 106 is configured to fit within a cartridge (insertion) compartment opening 108 in top housing 104-2 (described below) and a channel extending through top housing 104-2 of device 104 as shown in FIG. 3. The cartridge (insertion) compartment opening 108 in the top housing 104-2 enables access for the cartridge assembly 106 so it can drive integrated needle 104-6 into the subcutaneous tissue of a user.

Upon needle insertion, opening 108 is closed off, so the housing provides some sealing properties and creates a continuous surface as shown in FIG. 2.

Detachable activation mechanism 102 compartment is outside of the hermetically sealed compartment that contains the pump and electronics of device 104 for delivering insulin so water ingress can occur without affecting device 104 performance. The amount of water ingress is limited by minimizing the volume of empty space in order to reduce the wetting nuisance that can occur after a user's swim or shower.

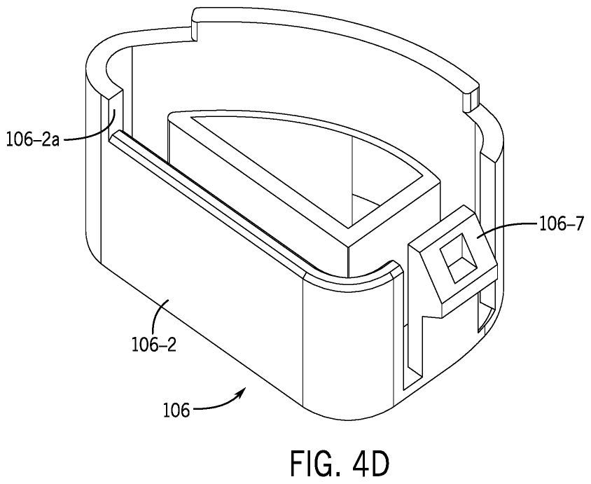

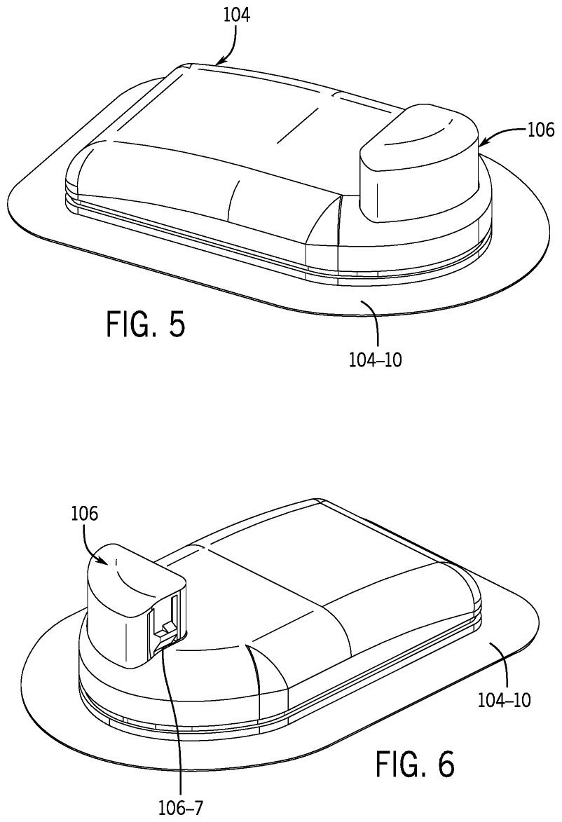

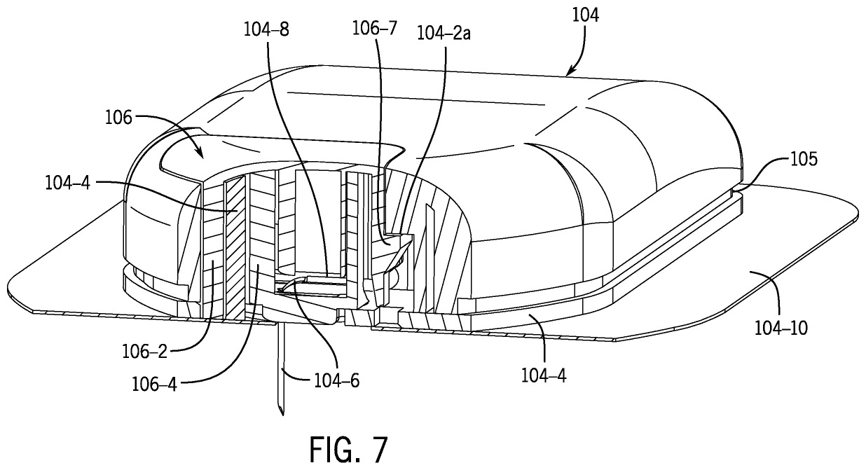

In brief, needle cartridge assembly 106 is configured to move from (1) a telescoping position above the top surface of device 104, as shown in FIGS. 5 and 6, wherein integrated infusion needle and CGM sensor 104-6 is in a retracted position to (2) an advanced position within device 104, i.e., above the user's tissue, as shown in FIGS. 2 and 7, wherein the infusion needle 104-6 is in a deployed position embedded into the subcutaneous layer of the user's tissue (after insertion). This is described in more detail below.

Device 104 for delivering insulin includes top housing 104-2, baseplate 104-4, integrated infusion needle and CGM sensor 104-6, insertion mechanism tubing 104-8 and adhesive patch 104-10. Device 104 includes groove 105 along the lower periphery thereof that is defined by housing 104-2 and baseplate 104-4 in an assembled configuration. Groove 105 is used to receive flanges of the components of detachable activation mechanism 102 as described in more detail below.

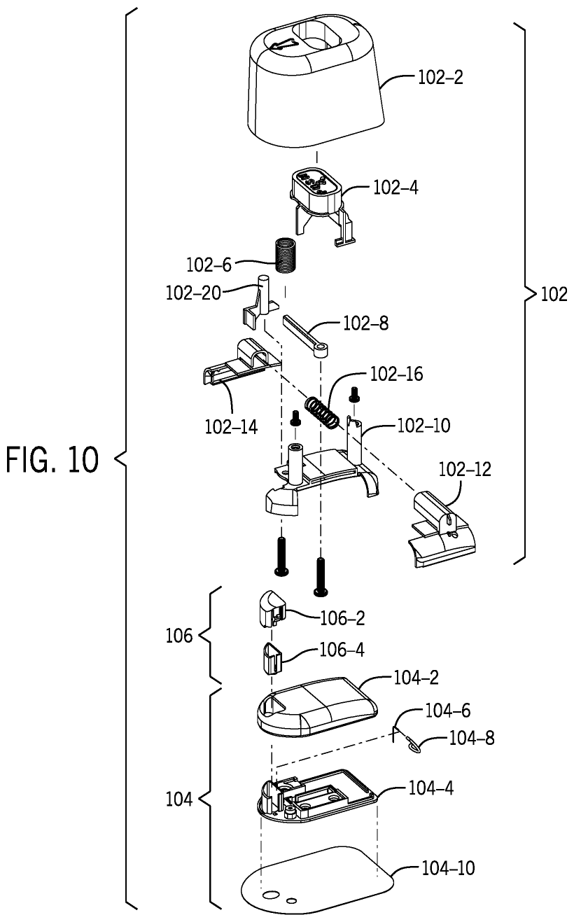

Detachable activation mechanism 102 includes detachable mechanism housing 102-2, activation button subassembly 102-4, insertion spring 102-6, activation beam 102-8, insertion mechanism inner housing 102-10, left release arm 102-12, right release arm 102-14, release spring 102-16, and a number of screws for assembling these components (shown but not numbered). These components are seen clearly in FIG. 10.

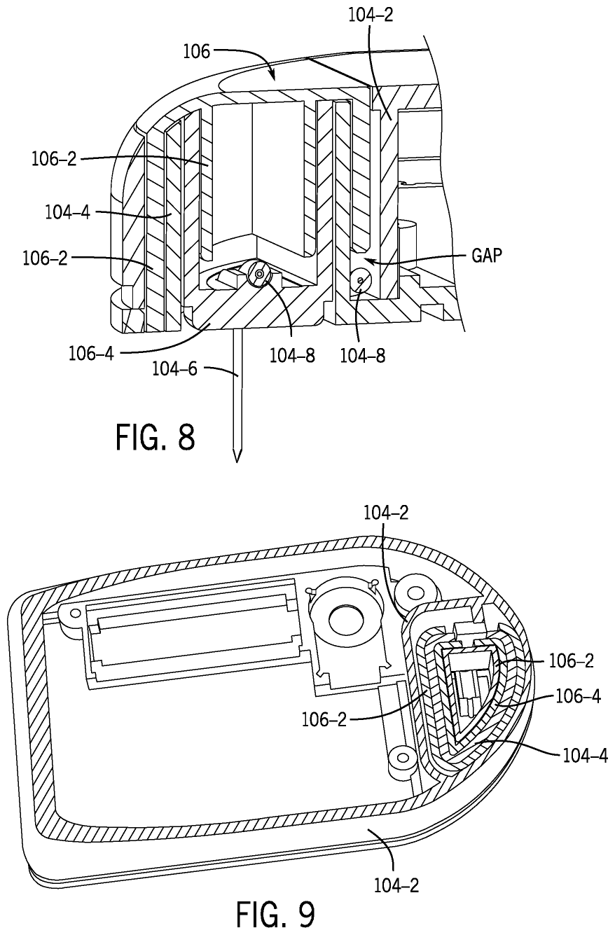

Needle cartridge assembly 106 includes needle cartridge 106-2, needle cartridge cover or insert 106-4 and needle cartridge lock snap (hook) 106-7. Needle cartridge cover 106-4 slides within needle cartridge 106-2 and is fully integrated together as best shown in FIG. 4B. The bottom of cover or insert 106-4 functions to actually cover needle cartridge assembly 106 as shown and described in more detail below. Integrated needle 104-6 is press fit through an opening in needle cartridge cover 106-4 to extend beyond the bottom surface thereof. This is best shown in FIGS. 4B, 4C and FIG. 8. Tubing 104-8 is fluidly connected to integrated infusion needle 104-6 as known to those skilled in the art and tubing 104-8 extends through side rear wall of cartridge cover 106-4 for fluid connection to a filled reservoir via a micropump. Lock snap 106-7 is configured to act as part of a locking mechanism to lock the cartridge assembly 106 down and force integrated infusion needle 104-6 in a user's skin. A ledge of a window within a wall of cartridge assembly 106. Specifically, needle cartridge lock snap 106-7 engages with a coupling ledge 104-2a (as the other part of the locking mechanism) as part of top housing 104-2 as seen in FIG. 7.

As indicated above, needle cartridge assembly 106, including cartridge 106-2, is configured to slide or move through a channel within device 104 from a telescoping position to a position fully integrated within device 104 whereby integrated infusion needle 104-6 is inserted into the subcutaneous tissue of the user. It is the construction of slidable needle cartridge assembly 106 and its operation with respect to device 104 offers the benefit of or facilitates a low profile configuration of device 104 while creating a hermetic seal to prevent leaks around the channel. To this end, needle cartridge assembly 106 is configured to slide through opening 108 and a channel defined by a wall in baseplate 104-4 and corresponding wall in top housing 104-2 (FIG. 8 for example). Specifically, baseplate 104-4 includes a planar surface or floor (wall) and a wall extending perpendicular to that floor (wall). The perpendicular wall creates a compartment for receiving needle cartridge assembly 106. FIGS. 8 and 9 best illustrate the wall structure and interaction with needle cartridge assembly 106 and baseplate 104-4.

Needle cartridge assembly 106 is configured with dual spaced guide walls that are defined by needle cartridge 106-2 and needle cartridge cover 106-4 as fully assembled. Upon activation, perpendicular wall of baseplate 104-4 slides within the gap between dual spaced guide walls defined by needle cartridge 106-2 and needle cartridge cover 106-4. Note that the distal end of outer wall of needle cartridge 106-2 is configured as uneven in length (not constant), thereby creating a step section 106-2a (as best seen in FIG. 4D) in that wall or an opening section (shorter in length) or gap between the wall and baseplate 104-4 when needle cartridge assembly 106 is fully advanced into device 104. This gap ensures that this outer wall does not interfere or compress tubing 104-8 during operation. This is shown in FIG. 8.

As can be seen in FIG. 9, the configuration of wall of top housing 104-2 that surrounds the dual walls of needle cartridge assembly 106 functions as a barrier to prevent any fluid seepage into remaining compartments of device 104 to protect other interior components such as a battery, power controller or MCU. That is, the wall structure (interaction and layering) of housing 104-2 in addition to the walls of needle cartridge assembly 106 as well as baseplate 104-4 help create a sealed compartment to function as the barrier to prevent fluid leakage into other compartments of device 104.

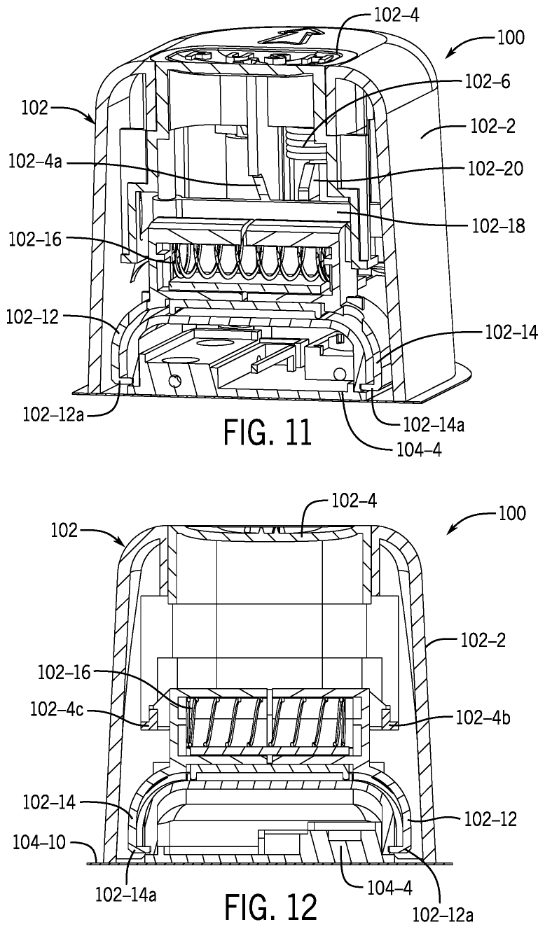

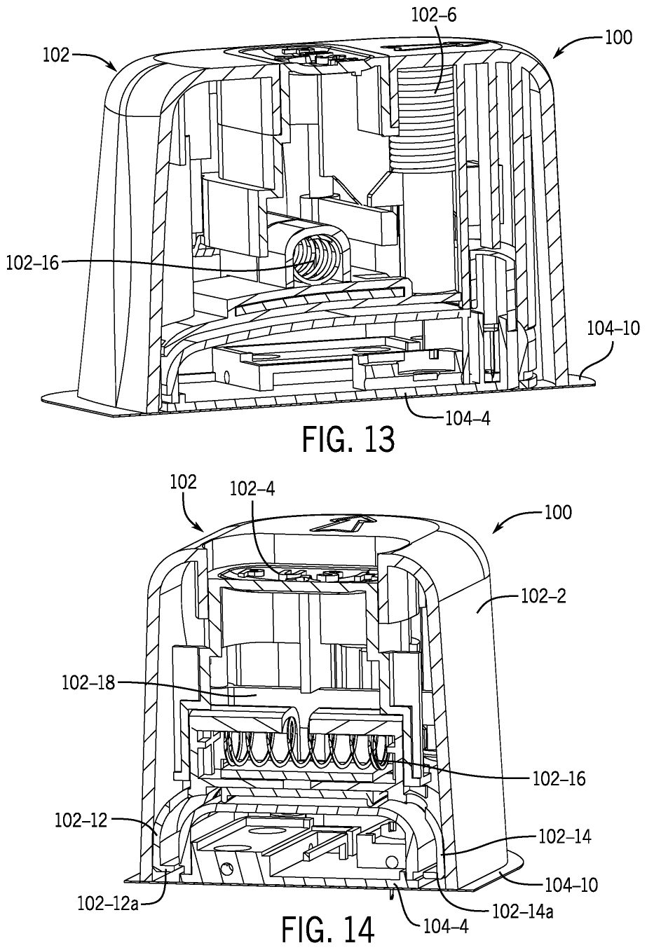

FIGS. 11-13 depict various cross-sectional views of the infusion system of FIG. 1 in a pre-activation configuration. In this configuration, device 104 for delivering insulin is securely attached to and within the detachable activation mechanism 102 by way of flanges 102-12a, 102-14a on right and left release arms 102-12, 102-14, respectfully. These flanges are biased outwardly by release spring 102-16, as shown in these FIGS. 11-13. In the locked position, flanges 102-12a, 102-14a engage groove 105 of device 104 to secure device 104 within detachable activation mechanism 102. Activation beam 102-8 holds insertion rod 102-20 in an upward position and spring 102-6 loaded. Button features or ledges 102-12b, 102-14b lock the release arms 102-12, 102-14 in the closed position so that flanges 102-12a, 102-14a within groove 105 lock the device 104 in detachable activation mechanism 102. Button ledges 102-12b, 102-14b interfere with release arms 102-12, 102-14 before activation.

When activation button assembly 102-2 is pressed, the ramped surface 102-4a on button assembly 102-4 translates downward, causing the activation beam 102-8 to rotate, freeing the release of insertion rod 102-20. Under load from spring 102-6, insertion rod 102-20 moves downward, thereby forcing cartridge assembly 106 downward as well. Insertion rod 102-20 pushes infusion needle 104-6 into an inserted position within the subcutaneous tissue layer of the user. Simultaneously, when button assembly 102-4 translates downward, button ledges 102-4b, 102-4c move into a position without interference from the release arms and spring 102-16 causes flanges 102-12a, 102-14b of release arms 102-12, 102-14 to move outwardly, thereby moving flanges 102-12a, 102-14a out of a groove 105 (also called indentation), releasing device 104.

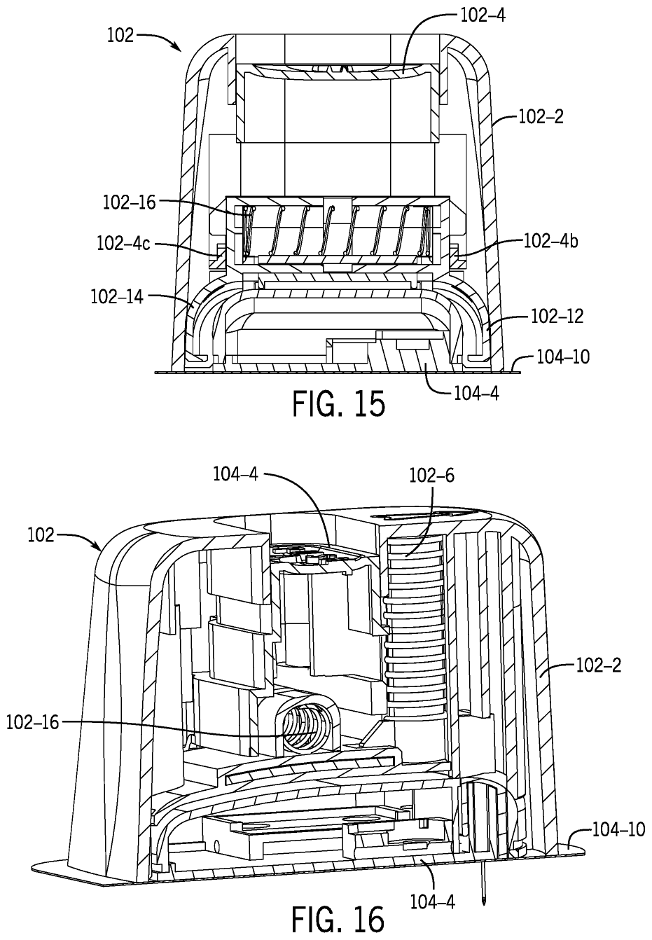

FIGS. 14-16 depict various cross-sectional views of the infusion system of FIG. 1 in a post activation configuration, but before detachable actuation mechanism 102 has been removed. Retention flanges 102-12a, 102-14a have moved outwardly, allowing device 104 to detach from detachable actuation mechanism 102.

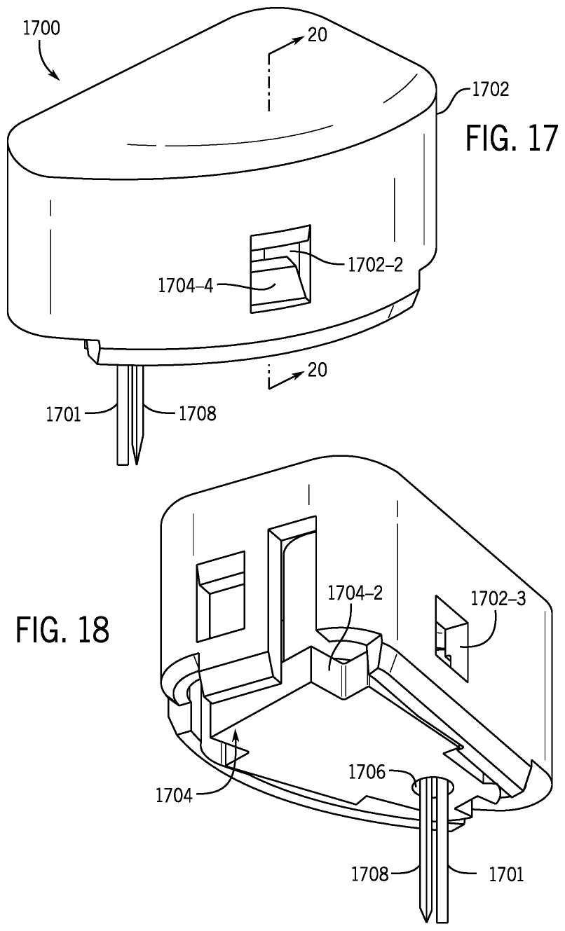

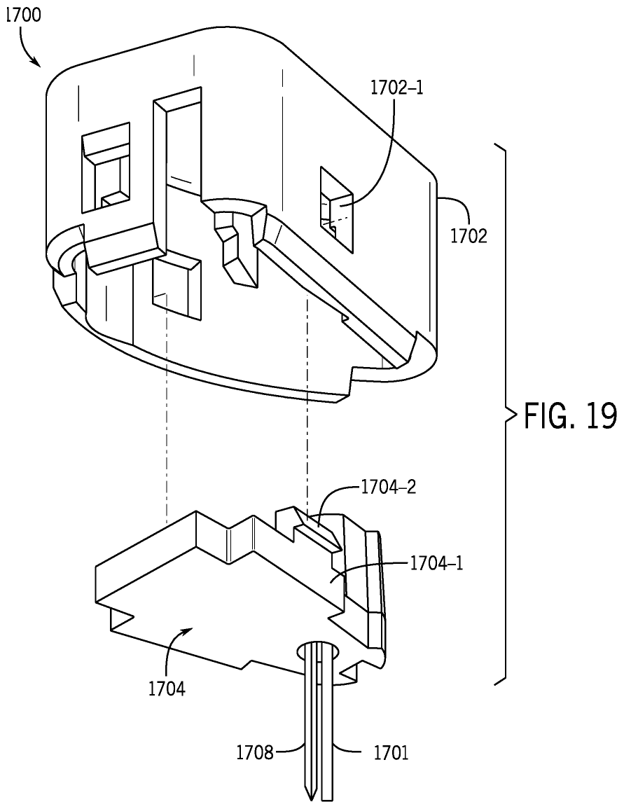

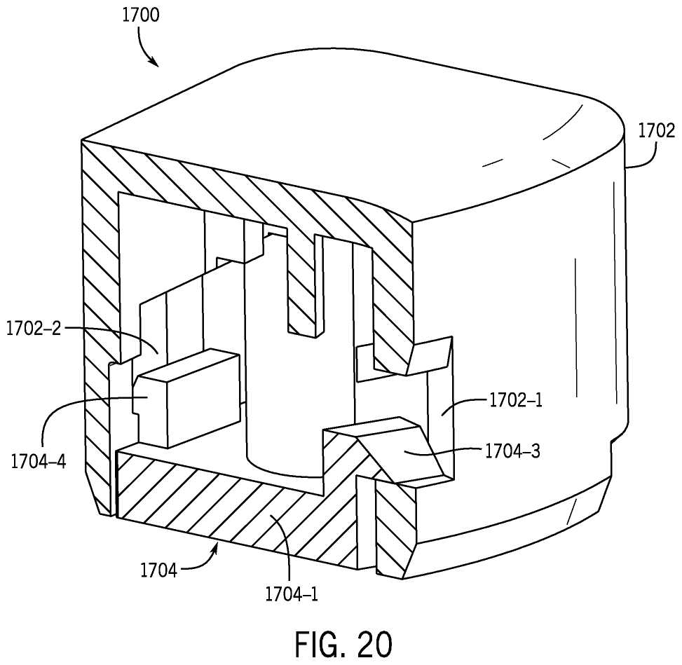

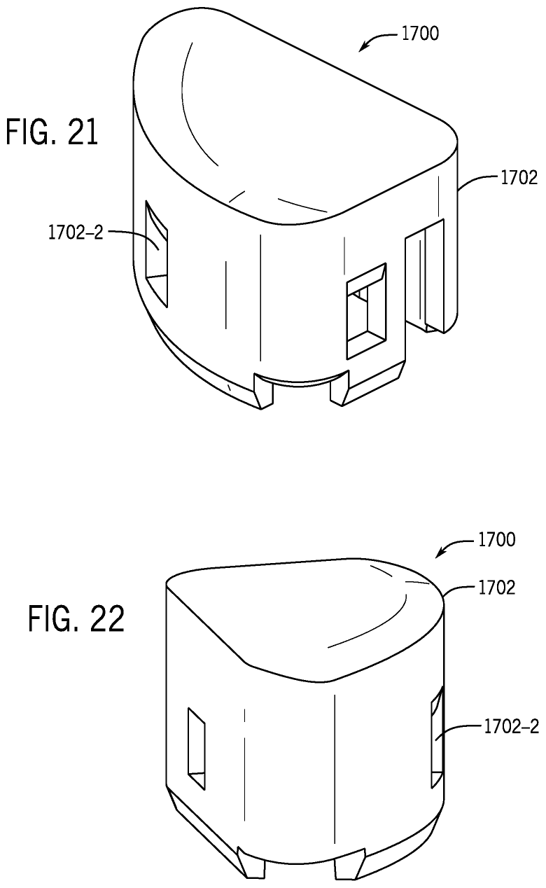

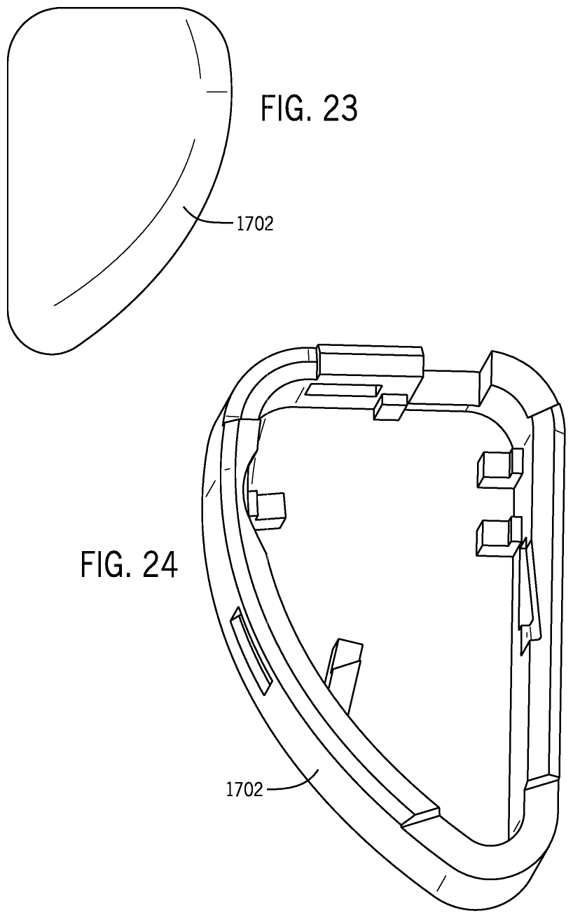

FIGS. 17-24 depict various views of another example needle cartridge assembly 1700 for an infusion system. In particular, cartridge assembly 1700 includes needle cartridge 1702 (also referred to as cartridge 1702) and cartridge cover/insert 1704. In this example, cartridge insert 1704 incorporates a CGM sensor (thus cartridge insert 1704 may be referred to as CGM cartridge insert 1702 in this example). In brief, CGM cartridge insert 1704 incorporates a CGM sensor 1701, electronics packaging to make electrical connections to its electrodes and a chassis to hold everything together. Cartridge insert 1702 incorporates a hole 1706 that can be used for introducer and/or infusion needle 1708 to perform successful insertion of CGM sensor 1701 into a user. By this construction, infusion needle 1708 may thus be integrated and held into place by cartridge insert 1704. As a result of this integration, a single point of insulin delivery and glucose sensing may take place in the interstitial fluid of the user (patient). This is described in more detail below. However, as an alternative to an infusion needle 1708, infusion system 100 may include one or more introducer needles for an infusion catheter, an analyte sensor such as a CGM sensor described herein or other medical component.

Specifically, in construction, needle cartridge assembly 1700 is defined by guide walls of needle cartridge 1702 and bottom of CGM cartridge insert 1704 as fully assembled (discussed below). Similar to other examples described herein, device 104 thus integrates infusion needle 1708 and CGM sensor 1704-1.

CGM cartridge insert 1704 is configured as a single base plate construction (described below) for supporting the integrated infusion needle 1708 and CGM sensor 1704-1 that is insertable within needle cartridge 1702. CGM cartridge insert 1704 houses and secures CGM sensor 1704-1 along with infusion needle 1708 within insert 1704 similar to the examples described above. Needle 1708 is configured to be fluidly connected to tubing as known to those skilled in the art and the tubing extends through side rear wall of cartridge insert 1704 for fluid connection to a filled reservoir via a micropump.

CGM cartridge insert 1704 also includes a base plate 1704-2 that functions as floor or cover for the bottom of cartridge assembly 1700. CGM cartridge insert 1704 actually inserts and snaps or rest in place within cartridge 1702 in a secured configuration using opposing locking hooks against internal ledges within the interior of cartridge 1702 as described in detail below. Specifically, CGM cartridge insert 1704 further includes two opposing locking hooks 1704-3, 1704-4 that extend vertically from base plate 1704-2 as shown. Locking hooks 1704-3, 1704-4 are configured to act as part of a locking mechanism to lock CGM cartridge insert 1704 within cartridge 1702 so as to enable integrated infusion needle 1708 and CGM sensor 1704-1 to puncture a user's skin when needle cartridge assembly 1700 is in a deployed configuration. Hooks 1704-3, 1704-4 engage with ledges of locking windows of cartridge 1702, as the other part of the locking mechanism as described in more detail below.

Needle cartridge 1702 further includes rectangular holes or windows 1702-1, 1702-2 that correspond in position to the position of hooks 1704-3, 1704-4 when cartridge insert 1704 is fully inserted into needle cartridge 1702 in an secured configuration. Each window is configured with a ledge for supporting or securing hooks 1704-3, 1704-4 within needle cartridge 1702. Hooks 1704-3, 1704-4 are part of a latching mechanism on cartridge insert 1704.

As indicated above in other example cartridge assemblies, needle cartridge assembly 1700, and hence cartridge 1702, is configured to slide or move through a channel 108 within device 104 from a telescoping position to a position fully integrated within device 104 whereby (integrated) infusion needle 1708 and CGM sensor 1704-1 are inserted into the subcutaneous tissue of the user. It is the construction of slidable needle cartridge assembly 1700 and its operation with respect to device 104 that offers the benefit of or facilitates a low profile configuration of device 104 while creating a hermetic seal to prevent leaks around the channel. Similar to the examples above, needle cartridge assembly 1700 is configured to slide through opening 108 and a channel defined by a wall in baseplate 104-4 and corresponding wall in top housing 104-2 (FIG. 8 for example). Specifically, baseplate 104-4 includes a planar surface or floor (wall) and a wall extending perpendicular to that floor (wall). The perpendicular wall creates a compartment for receiving needle cartridge assembly 1700.

Similar to the example above, needle cartridge assembly 1700 includes a needle cartridge lock snap (hook) and needle cartridge cover 1700 slides within needle cartridge 1702 (similar to that fully integrated together as shown in FIG. 4B). As shown and described, needle cartridge assembly 1700 incorporates a CGM sensor and infusion/introducer needle 1708 or catheter but may incorporate a CGM sensor or an infusion needle (alone) as known to those skilled in the art.

With the design of this cartridge assembly, the needle cartridge assembly is suitable to integrate any cartridge insert with a CGM sensor (including commercially available cartridge inserts for CGM sensing (sensor) along with an infusion needle or catheter).

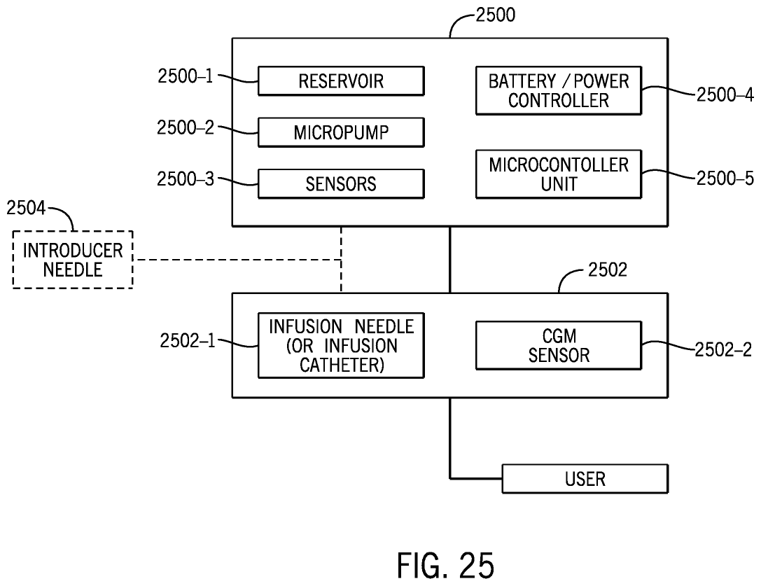

FIG. 25 depicts a block diagram of example components of (1) device 2500 for delivering insulin and (2) cartridge assembly 2502 of infusion system 100 as described in detail above. Specifically, device 2500 incorporates several components or modules (not shown) in the fluidic pathway including reservoir 2500-1 for storing the insulin, micropump 2500-2 for pumping the insulin, sensors 2500-3 (e.g., pressure) for sensing various parameters in the system and user and tubing connecting infusion needle 2502-1 to reservoir 2500-1 within cartridge assembly 2502. Device 2500 also includes microcontroller unit (MCU) 2500-4 and battery and power controller 2500-5. Cartridge assembly 2502 also includes CGM sensor 2500-5. CGM, as known to those skilled in the art, tracks user glucose levels and permits those levels to be used in algorithms that control flow rate. MCU 2500-4 controls the operation of micropump 2500-2. Infusion needle 2502-1 and CGM sensor 2502-2 are shown as separate components in FIG. 25 for illustration purposes. Infusion needle 2502-1 and CGM sensor 2502-2 may be integrated as shown in the examples above or the include infusion needle 2502-1 or CGM sensor may be used individually (without the other) in other infusion systems. Alternatively, an introducer needle 2504 may be used to introduce an infusion catheter and a CGM sensor as desired.

Reservoir 2500-1 is configured to receive and store insulin for its delivery over a course of about three days, or as needed. However, reservoir size may be configured for storing any quantity of fluid as required.

MCU 2500-5 electronically communicates with sensors 2500-3 and micropump 2500-2 as well as the CGM sensor 2502-2, as the monitoring components. Among several functions, MCU 2500-5 operates to control the operation of micropump 2500-2 to deliver insulin through insulin needle 2502-1 from reservoir 2500-1 at specific doses, i.e., flow rates over specified time intervals, based on CGM data converted to desired flow rate via control algorithms.

Battery and power controller 2500-4 controls the power to MCU 2500-5 and micropump 2500-2 to enable those components to function properly as known to those skilled in the art. CGM sensor 2500-2 is powered by battery and power controller 2500-4 through MCU 2500-5.

The components of device 2500 and cartridge assembly 2502 shown in FIG. 25 are only a few components. Those skilled in the art know that device 2500 and cartridge assembly 2502 include additional components.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. An infusion system comprising:
a device for delivering fluid to a user, the device including a reservoir and a micropump for pumping the fluid from the reservoir into a user; and
a cartridge for supporting a cartridge insert that includes (a) an infusion needle configured to infuse the fluid and introduce an analyte sensor into the user or (b) an introducer needle for introducing an infusion catheter and the analyte sensor, the cartridge configured to move from (1) a first position, wherein the infusion needle or an introducer needle is in a non-deployed position outside of the user to (2) a second position, wherein the infusion needle or an introducer needle is in a deployed position inserted into tissue of the user,
wherein the cartridge includes a locking mechanism to lock the cartridge insert into the cartridge assembly.

2. The infusion system of claim 1 wherein the locking mechanism includes a one or more windows for receiving a latching mechanism on the cartridge insert.

3. The infusion system of claim 2 wherein the one or more windows each includes a ledge for engaging a hook of the latching mechanism.

4. The infusion system of claim 1 further comprising a detachable activation mechanism, engaging the device for delivering insulin, for moving the cartridge from the first position to the second position, thereby releasing the device for delivering fluid.

5. The infusion system of claim 1 wherein the fluid is insulin.

6. The infusion system of claim 1 wherein the analyte sensor is a CGM sensor.

7. The infusion system of claim 1 further comprising the cartridge insert.

8. An infusion system comprising:
a device for delivering insulin to a user, the device including a reservoir and a micropump for pumping the insulin from the reservoir into tissue of a user; and
a cartridge for securing a cartridge insert that includes (a) an infusion needle configured to infuse the insulin and introduce a CGM sensor into the user or (b) an introducer needle for introducing an infusion catheter and the CGM sensor into the user, the cartridge configured to move from (1) a first position, wherein the infusion needle or an introducer needle is above the tissue of the user to (2) an second position, wherein the infusion needle or the introducer needle is in a deployed position inserted into tissue of the user,
wherein the cartridge includes a locking mechanism to lock the cartridge insert into the cartridge.

9. The infusion system of claim 8 wherein the locking mechanism includes a one or more windows for receiving a latching mechanism on the cartridge insert.

10. The infusion system of claim 9 wherein the one or more windows each includes a ledge for engaging a hook of the latching mechanism.

11. The infusion system of claim 8 further comprising a detachable activation mechanism, engaging the device for delivering insulin, for moving the cartridge assembly from the first position to the second position, thereby releasing the device for delivering insulin.

12. The infusion system of claim 8 further comprising the cartridge insert

13. An infusion system comprising:
a device for delivering insulin to a user, the device including a reservoir and a micropump for pumping the insulin from the reservoir into tissue of a user; and
a cartridge assembly including:
(1) a cartridge insert that includes (a) an integrated infusion needle and CGM sensor or (b) an introducer needle for introducing an infusion catheter and a CGM sensor and
(2) a cartridge for securing therein the cartridge insert, the cartridge assembly configured to move from (a) a telescoping position, wherein the integrated infusion needle and CGM sensor or an introducer needle, infusion catheter and CGM sensor are in a retracted position above the tissue of the user to (b) an advanced position, wherein the integrated infusion needle and CGM sensor or an introducer needle, infusion catheter and CGM sensor is in a deployed position inserted into tissue of the user,
wherein the cartridge includes a locking mechanism to lock the cartridge insert into the cartridge.

14. The infusion system of claim 13 wherein the locking mechanism includes a one or more windows for receiving a latching mechanism on the cartridge insert.

15. The infusion system of claim 14 wherein the one or more windows each includes a ledge for engaging a hook of the latching mechanism.

16. The infusion system of claim 13 further comprising a detachable activation mechanism, engaging the device for delivering insulin, for moving the cartridge assembly from the telescoping position to the advanced position, thereby releasing the device for delivering insulin.
